(19) United States
(12) Patent Application Publication
(10) Pub. No.: US 2021/0275556 A1
(43) Pub. Date: Sep. 9, 2021

(54) Cannabidiol Combination Compositions

(71) Applicant: Companion Sciences. LLC, New York, NY (US)

(72) Inventors: Golan VAKNIN, Brooklyn, NY (US); Tami Bar, Meitar (IL)

(73) Assignee: Companion Sciences. LLC, New York, NY (US)

(21) Appl. No.: 17/257,244

(22) PCT Filed: Jul. 1, 2019
(86) PCT No.: PCT/US2019/040043

§ 371 Date: Dec. 30, 2020

Related U.S. Application Data
(60) Provisional application No. 62/692,865, filed on Jul. 2, 2018.

(51) Int. Cl. A61K 31/7008; A61K 31/047; A61K 31/737; A61K 31/10; A61K 31/19; A61P 19/02

(52) U.S. Cl. CPC A61K 31/7008; A61K 31/047; A61P 19/02; A61K 31/10; A61K 31/19; A61K 31/737

(57) ABSTRACT

Embodiments of the invention are directed to formulations comprising Cannabidiol (CBD) and glucosamine (Gln). Further embodiments of the invention are directed to methods of treating or preventing Arthritis, Osteoarthritis, Rheumatoid arthritis, Osteoporosis, Osteopenia, jaw pain, joint pain, knee pain, back pain, multiple sclerosis, Osteomalacia and Paget's disease of bone, wherein the method comprises administering a formulation comprising Cannabidiol (CBD) and glucosamine (Gln).

### CROSS REFERENCE TO RELATED APPLICATIONS

This application claims the benefit of U.S. Provisional Application No. 62/692,865, filed on Jul. 2, 2018, which is incorporated in its entirety herein by reference.

### FIELD OF THE INVENTION

The current disclosure relates generally to formulations comprising Cannabidiol (CBD) and glucosamine (Gln) and methods of treatment.

### BACKGROUND OF THE INVENTION

Osteoarthritis (OA) is the most common form of arthritis, affecting millions of people worldwide. It occurs when the protective cartilage that cushions the ends of your bones wears down over time.

Although osteoarthritis can damage any joint, the disorder most commonly affects joints in the hands, knees, hips and spine.

Osteoarthritis is a common disease in small animals, as it is in humans. It has been estimated that around 30-50% of dogs and cats will be affected by osteoarthritis at some point in their lives (according to Willows Veterinary Centre and Referral Service).

Compositions comprising Glucosamine, Glucosamine and Chondroitin, or Glucosamine, Chondroitin sulfate and Methylsulfonylmethane (MSM) (this complex is known commercially as Mega Gluflex) are used in order to protect and heal deterioration of cartilage between joints, e.g., in osteoarthritis.

In many human and animal trials, these compositions and complexes, although found to be safe, are not found to be effective and have little or no improvement in either pain relief or joint damage.

Cannabis produces a variety compounds known as cannabinoids, many of which have not been detected in any other plant. Several cannabinoids were proved to have beneficial medical effect, including tetrahydrocannabinol (THC) and cannabidiol (CBD). Unlike THC, CBD does not appear to have any intoxicating effects. CBD is currently being used for various diseases and disorders with/without THC.

### SUMMARY OF THE INVENTION

In one aspect the invention relates to a composition comprising Cannabidiol (CBD) and Glucosamine, and/or a pharmaceutically acceptable glucosamine salt, in a pharmaceutically acceptable dosage form. In an embodiment of the invention, the glucosamine is in the form of a pharmaceutically acceptable salt selected from the group consisting of glucosamine sulfate, glucosamine hydrochloride and/or N-acetyl-glucosamine. In an embodiment of the invention, the glucosamine is in the form of glucosamine sulfate. In an embodiment of the invention, the CBD is extracted from a plant source. In an embodiment of the invention, the CBD is synthetic or semi-synthetic. In an embodiment of the invention, the composition, further comprises at least one additional active ingredient. In an embodiment of the invention, the at least one additional active ingredient is selected from the group comprising: Chondroitin, MSM, Boswellia serrata extract (Aflapin) or a combination thereof. In an embodiment of the invention, the CBD increases the bioavailability of the Glucosamine. In an embodiment of the invention, the CBD increases the bioavailability of the at least one active ingredient. In an embodiment of the invention, CBD and glucosamine have a complementary synergetic effect. In an embodiment of the invention, the CBD and the at least one additional active ingredient have a complementary synergetic effect.

In another aspect, the invention relates to a composition comprising Cannabidiol (CBD) and Glucosamine, and/or a pharmaceutically acceptable glucosamine salt, in a pharmaceutically acceptable dosage form, for use in the treatment of at least one disease, condition, symptom or disorder associated with bone and joint diseases. In an embodiment of the invention, the at least one disease is osteoarthritis.

In another aspect, the invention relates to a method of treating a disease, condition, symptom or disorder associated with bone and joint diseases in a subject in need thereof; said method comprising administration of a therapeutically effective amount of a composition comprising Cannabidiol (CBD) and Glucosamine and/or a pharmaceutically acceptable glucosamine salt, in a pharmaceutically acceptable dosage form. In an embodiment of the invention, the disease is osteoarthritis. In an embodiment of the invention, the glucosamine is glucosamine sulfate. In an embodiment of the invention, the CBD is extracted from a plant source. In an embodiment of the invention, the CBD is synthetic or semi-synthetic. In an embodiment of the invention, the composition further comprises at least one additional active ingredient. In an embodiment of the invention, the at least one additional active ingredient is selected from the group comprising: Chondroitin, MSM, Boswellia serrata extract (Aflapin) or a combination thereof. In an embodiment of the invention, the CBD is administered simultaneously as the glucosamine. In an embodiment of the invention, the CBD is administered separately from the glucosamine.

In another aspect, the invention relates to a method of increasing bioavailability of glucosamine in a therapeutic formulation containing an effective amount of glucosamine for treatment of at least one disease, condition, symptom or disorder associated with bone and joint diseases, the method comprising administering Cannabidiol (CBD) in the formulation in a predetermine ratio with the glucosamine. In an embodiment of the invention, the CBD is administered simultaneously as the glucosamine. In an embodiment of the invention, the CBD is administered separately from the glucosamine. In an embodiment of the invention, the at least one disease is osteoarthritis. In an embodiment of the invention, the glucosamine is in the form of a pharmaceutically acceptable salt selected from the group consisting of glucosamine sulfate, glucosamine hydrochloride and/or N-acetyl-glucosamine. In an embodiment of the invention, the glucosamine is in the form of glucosamine sulfate. In an embodiment of the invention, the CBD is extracted from a plant source. In an embodiment of the invention, the CBD is synthetic or semi-synthetic.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 depicts a graph with data from Example 1 compering the levels of plasma Glucosamine of the different compositions one hour, four hours and eight hours after administration.

FIG. 2 depicts a graph with data from Example 1 comparing the levels of plasma CBD of the different compositions one hour, four hours and eight hours after administration.

FIG. 3 depicts a graph with data from Example 1 comparing the levels of Synovial Fluid Glucosamine of the different compositions eight hours after administration.

FIG. 4 depicts a graph with data from Example 1 comparing the levels of CBD plasma concentration of the different compositions over time.

FIG. 5 is a picture of dog treats from Example 2.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

Embodiments of the invention are directed to formulations comprising Cannabidiol (CBD) and glucosamine (Gln). The combination of CBD and glucosamine may be referred to in the application as “the combination” and/or “the formulation”.

As used herein, “glucosamine” includes glucosamine formulated as a pharmaceutically acceptable salt, including without limitation glucosamine sulfate, glucosamine hydrochloride and N-acetyl-glucosamine.

Further embodiments of the invention are directed to methods of treating or preventing bone and joint diseases, conditions, or symptoms. In some embodiments the bone and joint diseases, conditions or symptoms are selected from: Arthritis, Osteoarthritis (OA), Rheumatoid arthritis, Osteoporosis, Osteopenia, jaw pain, joint pain, knee pain, back pain, multiple sclerosis, Osteomalacia and Paget's disease of bone, wherein the method comprises administering a formulation comprising Cannabidiol (CBD) and glucosamine (Gln) and/or a pharmaceutically acceptable glucosamine salt, in a pharmaceutically acceptable dosage form.

In some embodiments the composition may provide pain relief for patients with osteoarthritis of the knee, hip or spine.

In some embodiments the composition may reduce pain related to rheumatoid arthritis.

In some embodiments the method may be used to treat humans or animals. Bothe humans and animals may be referent to as “patients” in the application.

The present invention relates to the surprising discovery that the combination of CBD and glucosamine increases the penetration of glucosamine into the synovial fluid and consequently increasing glucosamine concentration in the synovial fluid. The combination itself of CBD and glucosamine may provide more efficient treatment and in addition the increased concentration of glucosamine may provide more effective treatment.

This discovery may improve treatment as one of the current treatments comprises administering, orally, a complex which includes glucosamine, chondroitin sulfate and MSM (Methyl sulfonyl methane); it has been shown that despite the achieved plasma and synovial fluid levels the concentrations were still too low to have a relevant biological effect on articular cartilage.

The combination may provide more effective treatment in multiple ways and levels.

The patients may benefit from the additive effects of each component. CBD has several known physiological effects. CBD may relive pain, reduce anxiety and depression, act as an anti-inflammatory agent, etc. Gln may support the structure and function of joints. In addition, Gln may increase the cartilage and fluid surrounding joints and may help prevent breakdown of these substances.

The patients may benefit from the complementary effects of each component. Anxiety and depression are interrelated with pain and physical limitation, which are well known OA symptoms and may relate to symptoms of other bone and joint diseases. Anxiety and depression can significantly impair quality of life of patients by altering pain perception and functional capacity. Accordingly, relief of pain and anxiety by administering CBD may allow faster and more efficient healing with Gln administration.

The patients may benefit from the co-operative effects of CBD and glucosamine. In this case, the activity of the two components is strongly depend one on the other. For example, CBD may improve the plasma- or organ-bioavailability of Glucosamine and thus provide a sufficient concentration of glucosamine for effective treating. Or, CBD may reduce inflammation and by doing so allow the glucosamine to be more effective.

In some embodiments the glucosamine is formulated as a pharmaceutically acceptable salt of glucosamine, including without limitation glucosamine sulfate, glucosamine hydrochloride and/or N-acetyl-glucosamine. In some embodiments the glucosamine is in the form of glucosamine sulfate. In some embodiments the glucosamine is in the form of glucosamine hydrochloride.

In some embodiments the Glucosamine may be replaced by other amino sugar molecules such as Galactosamine, Sialic acid and N-Acetylglucosamine.

When referring to CBD it should be understood to encompass any enantiomer, diastereomer, or derivative thereof.

In some embodiments, CBD is extracted from a plant source. In other embodiments, the CBD is synthetic or semi-synthetic.

In some embodiments the CBD is water soluble CBD. In other embodiments the CBD is in the form of an oil. In other embodiments the CBD in in the form of oil-in-water. In other embodiments the CBD is in the form of crystals.

In some embodiments CBD and Gln are administered separately. In some embodiments the CBD is administered first followed by Gln administration. In some embodiments the Gln is administered first followed by CBD administration.

In some embodiments, the formulation further comprises one or more additional active ingredient(s). In some embodiments the additional active ingredient is administered separately.

In some embodiments the additional active ingredient is Chondroitin sulfate. In some embodiments the additional active ingredient is MSM (Methylsulfonylmethane). In some embodiments the additional active ingredients are Chondroitin sulfate and MSM. In some embodiment the additional active ingredient is Boswellia serrata extract (Aflapin). Boswellia serrata may possesses pharmacological properties like anti-arthritic, anti-inflammatory, analgesic and hepatoprotective that may offer relief in OA and/or in other bone and joint diseases. In some embodiment the additional active ingredients are MSM and Boswellia serrata extract. In some embodiment the additional active ingredients are Chondroitin sulfate and Boswellia serrata extract. In some embodiment the additional active ingredients are MSM and Chondroitin sulfate and Boswellia serrata extract.

In some embodiments the composition is used for treating bone and joint diseases, conditions, or symptoms. In some embodiments the bone and joint diseases, conditions or symptoms are selected from: Arthritis, Osteoarthritis, Rheumatoid arthritis, Osteoporosis, Osteopenia, jaw pain, joint pain, knee pain, back pain, multiple sclerosis Osteomalacia and Paget's disease of bone. In some embodiment the bone and joint disease is Osteoporosis.

In some embodiments the composition is used to treat humans. In some embodiments the composition is used to treat animals. In some embodiments the composition is used to treat pets. In some embodiments the composition is used to treat dogs. In some embodiments the composition is used to treat horses.

### Doses for Treating Humans

In some embodiments the daily CBD dose used to treat humans is represented in table 1 below (mg/Kg):

TABLE 1

Weight (Kg)

| Pain | <11 | 12-20 | 21-39 | 40-68 | 69-109 | >110 |
|---|---|---|---|---|---|---|
| Mild | 0.4 | 0.4 | 0.3 | 0.2 | 0.2 | 0.2 |
| Medium | 0.6 | 0.5 | 0.4 | 0.3 | 0.3 | 0.3 |
| Severe | 0.8 | 0.7 | 0.5 | 0.4 | 0.3 | 0.3 |

The doses may be adjusted according to the medical condition, time of day, mode of administration, formulation, composition with glucosamine sulfate, composition with THC, composition with additional ingredients (such as MSM and Chondroitin sulfate).

In some embodiments the daily glucosamine sulfate dose used to treat humans is about 500-2,000 mg. In some embodiments the daily glucosamine sulfate dose used to treat humans is about 1,500.

In some embodiments the daily MSM dose used to treat humans is about 250-1000 mg. In some embodiments the daily MSM dose used to treat humans is about 500.

In some embodiments the daily chondroitin sulfate dose used to treat humans is about 400-1500 mg. In some embodiments the daily chondroitin sulfate dose used to treat humans is about 1200.

In some embodiments the daily Boswellia serrate extract dose used to treat humans is about 250-1000 mg. In some embodiments the daily Boswellia serrate extract dose used to treat humans is about 500.

In some embodiments the concentration of Gln is in a range of about 10% to 100% w/w in the formulation. Wherein every unit weigh about 1-5 g, and the dose is from about 500 to about 2,000 mg/day.

In some embodiments the concentration of CBD is in a range of 0.1% to 15% w/w in the formulation. Wherein every unit weigh about 1-5 g, and the dose is from about 5 to about 150 mg/day.

In some embodiments the formulation may be administered orally. In some embodiments the formulation is administered orally for treating osteoarthritis at a dose of about 3000 mg (including both CBD and glucosamine) once daily or about 1000 mg three times daily.

In some embodiments the dose range is about 200-6000 mg/day (including both CBD and glucosamine).

In some embodiments the formulation is administered orally for treating osteoarthritis at a dose of about 1,500 mg Glucosamine Sulfate, about 100 mg CBD and about 1,000 mg Chondroitin sulfate.

In some embodiments the formulation is administered orally for treating osteoarthritis at a dose of about 500 mg Glucosamine Sulfate, about 200 mg CBD, about 400 mg Chondroitin sulfate and about 200 mg MSM.

In some embodiments the formulation may be applied to the skin. In some embodiments the formulation is applied to the skin for treating osteoarthritis wherein the formulation comprises about 30 mg/gram of glucosamine sulfate and about 50 mg/gram of CBD. In some embodiments the formulation comprises: about 30 mg/gram of glucosamine sulfate, about 50 mg/gram of CBD and about 140 mg/gram of chondroitin sulfate.

In some embodiments the formulation may be injected directly into the muscle. In some embodiment the formulation is injected directly into the muscle for osteoarthritis at a dose of about 400 mg (including both CBD and glucosamine).

In some embodiments the ratio of CBD:Gln is 1:1. In some embodiments the ratio is 1:2. In some embodiments the ratio is 2:1. In some embodiments the ratio is 1:3. In some embodiments the ratio is 1:5. In some embodiments the ratio is 1:10. In some embodiments the ratio is 1:15. In some embodiments the ratio is 1:20. In some embodiments the ratio is 1:30. In some embodiments the ratio is 1:40. In some embodiments the ratio is 1:50.

### Doses for Treating Animals

In some embodiments the daily CBD dose used to treat dogs is represented in table 2 below (mg/Kg):

TABLE 2

| Pain | Dose (mg/Kg) |
|---|---|
| Mild | 0.22 |
| Medium | 0.55 |
| Severe | 1.1 |

The doses may be adjusted according to the medical condition, time of day, mode of administration, formulation, composition with glucosamine sulfate, composition with THC, composition with additional ingredients (such as MSM and Chondroitin sulfate).

In some embodiments the doses are in the range of about 1/10 of the above.

In some embodiments the daily glucosamine sulfate dose used to treat dogs is about 22-44 mg/Kg. In some embodiments the daily MSM dose used to treat dogs is about 250-1,500 mg/Kg.

In some embodiments the daily Chondroitin sulfate dose used to treat dogs is about 250-1,500 mg/Kg. In some embodiments the daily Boswellia serrate extract dose used to treat dogs is about 100 mg/Kg.

In some embodiments the doses for treating dogs may be adjusted by weight and used for treating cats. In some embodiments the doses for treating dogs may be adjusted by weight and used for treating other animals. The doses for dogs and animals are determined according to the dog's/animal's weight and the severity of the disease.

In some embodiments the formulation is administered orally to dogs for treating osteoarthritis at a low dose of about 20 mg/Kg Glucosamine Sulfate, about 0.15 mg/Kg CBD, about 200 mg/Kg Chondroitin sulfate, about 200 mg/Kg MSM and about 5 mg/Kg of Boswellia serrate.

In some embodiments the formulation is administered orally to dogs for treating osteoarthritis at a high dose of about 50 mg/Kg Glucosamine Sulfate, about 2 mg/Kg CBD, about 2,000 mg/Kg Chondroitin sulfate, about 2,000 mg/Kg MSM and about 5 mg/Kg of Boswellia serrate.

In some embodiments the ratio of CBD:Gln is 1:1. In some embodiments the ratio is 1:2. In some embodiments the ratio is 2:1. In some embodiments the ratio is 1:3. In some embodiments the ratio is 1:5. In some embodiments the ratio is 1:10. In some embodiments the ratio is 1:15. In some embodiments the ratio is 1:20. In some embodiments the ratio is 1:30. In some embodiments the ratio is 1:40. In some embodiments the ratio is 1:50.

### Administration and Pharmaceutically Acceptable Dosage Forms

The compositions, or each ingredient, may be administered to a subject, human or animal, by any method known to a person skilled in the art, such as topically, parenterally, paracancerally, transmucosally, transdermally, intramuscularly, intravenously, intradermally, subcutaneously, intraperitonealy, intraventricularly, intracranially, intravaginally or intratumorally.

In some embodiments the composition, or each ingredient, is administered directly into the synovial fluid.

In some embodiments the composition, or each ingredient, is administered by vapor.

In some embodiments, the compositions, or each ingredient, may be added to food. In some embodiments the food is pet food. In some embodiments the food is edibles such as gummies.

The composition, or each ingredient, may be packed in liposomes or emulsions of collagen, collagen peptides or other components of skin or basement membrane.

The absorption of the ingredients may be increased by combining the use of hostile biophysical environments with the use of penetrating agents, such as, but not limited to, oleoresin capsicum or its constituents or molecules containing heterocyclic rings to which hydrocarbon chains are attached.

The compositions of the present invention may include additional ingredients that are not physiologically active but serve to enhance the properties of the final composition. For example, the compositions of the present invention may include excipients such as lactose, dextrose, sucrose, sorbitol, mannitol, starch, gum acacia, calcium silicate, microcrystalline cellulose, polyvinylpyrrolidinone, cellulose, water, syrup, and methyl cellulose. The compositions of the present invention may include lubricating agents such as talc, magnesium stearate and mineral oil, wetting agents, emulsifying and suspending agents, preserving agents such as methyl- and propylhydroxybenzoates, sweetening agents or flavoring agents.

The compositions of the present invention may be formulated in any pharmaceutically acceptable topical vehicle that does not interact adversely with the active ingredients. Compositions of the present invention may be formulated in water or oil based topical vehicles. These compositions, in some embodiments, can include lanolin, aquaphor, methylcellulose and derivatives thereof, petroleum based vehicles, Aloe vera and the like. In another embodiment, the compositions of the present invention are formulated in a topical, water-based vehicle containing Aloe vera and vitamin E.

The topical compositions of the present invention may include distilled water oil, stearic acid, an alcohol, an emulsifying wax, glycerin, palmitic acid, denatured alcohol, methyl salicylate, lecithin, sodium bicarbonate, ascorbyl palmitate, polysorbate, methylparaben, propylparaben, or any combination thereof. The topical compositions of the present invention may have a pH of between about 3 and about 8.

According to embodiments topical composition of the present invention are in the form of an ointment, a cream, a lotion, an oil, a solution (in some embodiments an aqueous solution), an emulsion, a gel, a paste and a milk. In some embodiments the carrier is an aqueous-based carrier (such as a gel, oil-in water emulsion or oil-in water cream, aqueous solution, foam, lotion, spray).

According to some embodiments, the composition is administered orally, wherein a unit dosage form used may comprise tablets, capsules, lozenges, chewable tablets, suspensions, emulsions and the like. Such unit dosage forms comprise a safe and effective amount of the desired compound, or compounds. The acceptable carrier suitable for the preparation of unit dosage forms for peroral administration are well-known in the art. Tablets typically comprise conventional pharmaceutically-compatible adjuvants such as inert diluents, such as calcium carbonate, sodium carbonate, mannitol, lactose and cellulose; binders such as starch, gelatin and sucrose; disintegrants such as starch, alginic acid and croscarmellose; lubricants such as magnesium stearate, stearic acid and talc. Glidants such as silicon dioxide may be used to improve flow characteristics of the powder-mixture. Coloring agents, such as the FD&C dyes, may be added for appearance. Sweeteners and flavoring agents, such as aspartame, saccharin, menthol, peppermint, and fruit flavors, may be useful adjuvants for chewable tablets, syrups, and the like. Capsules typically comprise one or more solid diluents disclosed above. The selection of carrier components depends on secondary considerations like taste, cost, and shelf stability, which are not critical for the purposes of this invention, and can be readily made by a person skilled in the art. In some embodiments the capsules are flavored-capsules.

The oral dosage form may include predefined release profile. In one embodiment, the oral dosage form of the present invention is an extended release formulation, formulated as extended release tablets, capsules, lozenges or chewable tablets. The oral dosage form of the present invention may comprise slow release tablets, capsules, lozenges or chewable tablets. The oral dosage form of the present invention may comprise immediate release tablets, capsules, lozenges or chewable tablets. The oral dosage form may be formulated according to the desired release profile of the active ingredients, as known to one skilled in the art.

Peroral compositions may comprise liquid solutions, emulsions, suspensions, and the like. The pharmaceutically-acceptable carriers suitable for preparation of such compositions are well known in the art.

Compositions for use in the methods of this invention may comprise solutions or emulsions, which, in some embodiments, are aqueous solutions or emulsions comprising a safe and effective amount of glucosamine and CBD and optionally, other compounds,

Other compositions may comprise dry powders. Compositions may be formulated for atomization and inhalation administration. Such compositions may be contained in a container with attached atomizing means.

### EXAMPLES

### Example 1—Penetration

Three different water-soluble CBD oral formulae were tested, F #01 (20%), L #02 (2.5% CBD) and I #03 (20% CBD), and administered at 15 mg/Kg rat.

The three formulae were tested in combination with 500 mg/Kg Glucosamine (Gln).

The total administered volume was 10 mL/Kg (CBD+Gln).

The trial was performed on Sprague Dawley male rat (n=3/group).

A single dose containing CBD and Gln was provided PO at t=0.

CBD and Gln plasma concentrations were examined 1, 4 and 8 hours following a single administration.

Gln concentration in the synovial fluid was examined 8 hours after oral administration.

### Results:

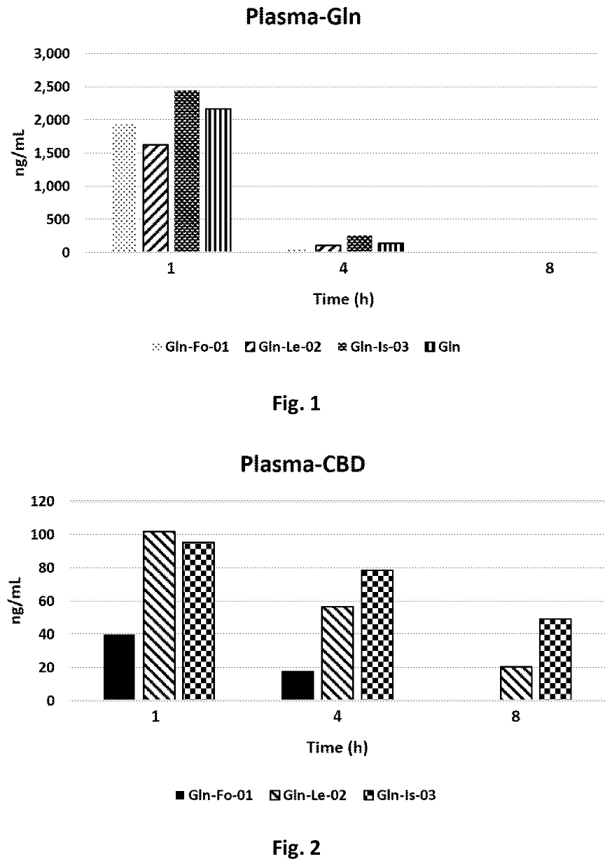

As can be seen in FIG. 1, No significant difference of Gln plasma concentration was found between the various groups.

One hour after administration, the average Gln plasma concentration was 2.039 ng/mL, decreased after 4 h to 134 ng/mL (a 93.4% decrease from base) and after 8 h to 8 ng/mL (a 99.6% decrease from base, and a 94.0% decrease from the 4 h point).

Following oral administration of 15 mg/Kg and 500 mg/Kg of CBD and Gln, respectively, no statistical significance was found in Gln plasma concentration between the three CBD water-soluble formulae tested, F #01, L #02 and I #03. This finding indicates that CBD did not significantly affect plasma Gln concentration. However, plasma concentration of Gln was lower for formula L #02.

On the other hand, as can be seen in FIG. 2, plasma CBD concentration was found to be different for the various formulae. In all time points, I #03 and L #02 presented the highest CBD plasma concentration. CBD plasma concentration following F #01 administration was significantly lower than that of I #03 and L #02. Despite the different values, it seems like the reduction rate for all three formulae is similar as could be observed from the scheme.

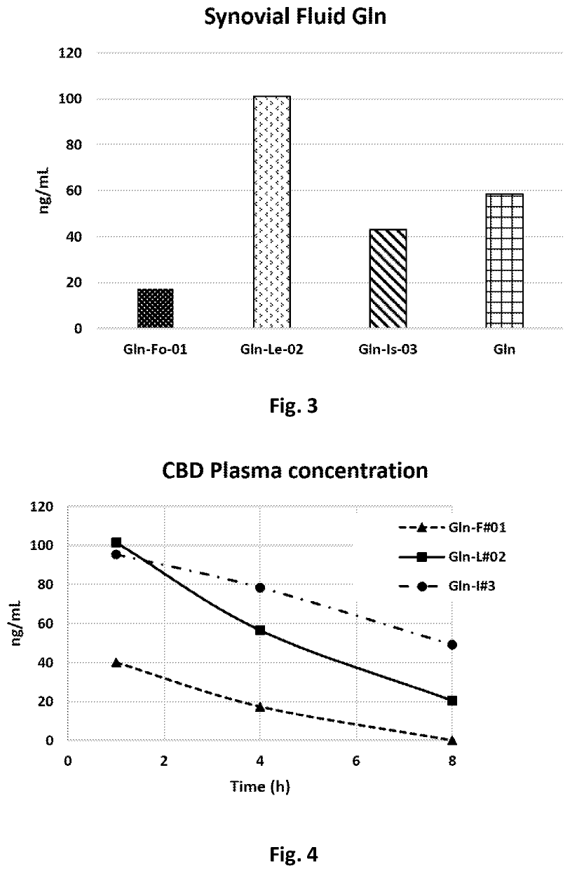

As can be seen in FIG. 3, 8 h after oral administration, Gln concentration in the synovial fluid was L #02>Gln=I #03>F #01.

Significant improvement of Gln concentration in the synovial fluid was observed only for L #02. This result is in agreement with the relatively low Gln plasma concentration observed for L #02.

Interestingly, following administration of F #01, Gln concentration in the synovial fluid was lower than that obtained for Gln per se. No CBD was detected in the synovial fluid.

The findings suggest that co-administration of oral water-soluble CBD with Gln results in increased Gln concentration in the synovial fluid.

### Example 2—Dog Snacks

Two compositions comprising CBD and Gln were formulated into dog snacks.

Composition A for small size dogs comprised 2.5 mg CBD and 400 mg Gln in a 6-gr snack.

Composition A for medium size dogs comprised 4.5 mg CBD and 700 mg Gln in a 10-gr snack.

Composition A for large size dogs comprised 7.5 mg CBD and 1.2 g Gln in 12-gr snack.

Composition B comprised 10 mg CBD and 1.5 g Gln.

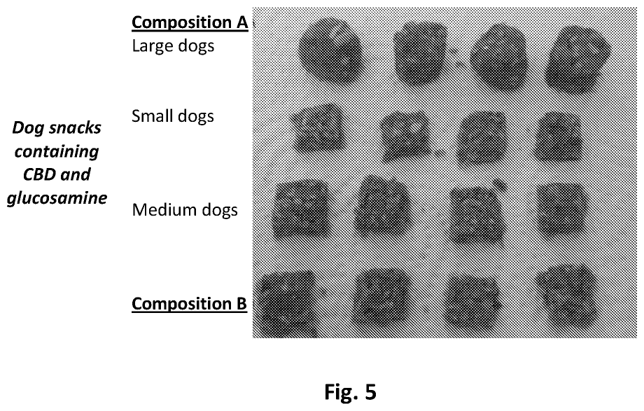

Each composition was formulated into dog snacks using soft dog snacks and flavoring agents. FIG. 5 is a picture of the dog snacks.

The snacks were administered to 5 healthy dogs.

The dogs ate the snacks easily showing that the dog snack formulation had a desirable taste and the bad taste of the CBD was masked.

### Example 3—Ratio

The trial includes approximately four animal groups (3 for synovial fluid and 1 for plasma) for four different CBD:Gln ratios (1:5, 1:10, 1:20 and 1:30) with two CBD formulae and one control (Gln alone). Each of these 48 groups holds 8 rats for a total of 384 animals (128 for plasma PK and 256 for synovial fluid).

The total dose is 10 mL/Kg.

The trial is performed on Sprague Dawley male rat (n=3/group).

A single dose is provided PO at t=0.

CBD and Gln plasma concentrations are examined 1, 4 and 8 hours following a single administration.

Synovial fluid is examined 8 hours after oral administration.

The target is to optimize the CBD:Gln ratio in order to achieve maximal Gln concentration in the synovial fluid while keeping acceptable and applicable oral doses of both CBD and Gln.

### Example 4—Treating Osteoarthritis

50 mice are given one unit of collagenase type VII intra-articularly into the right knee on days 0 and 2 to induce joint instability. The collagenase-induced OA model is a model based on induction of joint instability by unilateral intra-articular injection of collagenase.

Pain is used as an indicator in the OA models.

A formulation comprising Gln and CBD is tested.

75 mice are randomly divided into three groups (25 mice/group): Group 1 (n=25): is given the formulation comprising Gln, CBD, Chondroitin, MSM and Boswellia serrata extract, Group 2 (n=25): is given the formulation comprising Gln and CBD and Group 3 (n=25): is the control group and given Gln only.

The total administered volume is 10 mL/Kg and provided PO.

Following the onset of pain on day 20 from the induced OA, the mice are treated twice a week for four weeks.

Levels of pain are measured daily for four weeks.

Results will indicate reduced levels of pain in Group 1 and Group 2 compared to Group 3.

### Example 5—Doses for Treating Osteoarthritis

A clinical trial is conducted to determine the does ranges for the combination of CBD and Gln.

50 human patients with OA are treated with different dose ranges according to the table below. The doses are administered once a day orally for three months.

The WOMAC Osteoarthritis Index is used to assess the activity of the dose ranges.

Results will indicate favourable ratios for treating humans with OA.

The daily CBD is according to table 3 below (mg/Kg):

TABLE 3

| | Weight (Kg) | | |
|---|---|---|---|
| Pain | 40-68 | 69-109 | >110 |
| Mild | 0.2 | 0.2 | 0.2 |
| Medium | 0.3 | 0.3 | 0.3 |
| Severe | 0.4 | 0.3 | 0.3 |

The patients receiving the above CBD dose are divided into four groups, wherein group 1 receives a daily dose of Gln in the amount of 1:10 (CBD:Gln) relative to the CBD dose received according to table 3, group 2 receives a daily dose of 1:20 and group 3 a daily dose of 1:30. Group 4 is similar to Group 2 but with the addition of Chondroitin, MSM and Boswellia serrata extract.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A composition comprising Cannabidiol (CBD) and Glucosamine, and/or a pharmaceutically acceptable glucosamine salt, in a pharmaceutically acceptable dosage form.

2. The composition of claim 1, wherein the glucosamine is in the form of a pharmaceutically acceptable salt selected from the group consisting of glucosamine sulfate, glucosamine hydrochloride, N-acetyl-glucosamine and combinations thereof.

3. The composition of claim 1, wherein the glucosamine is in the form of glucosamine sulfate.

4. The composition according to claim 1, wherein the CBD is extracted from a plant source.

5. The composition according to claim 1, wherein the CBD is synthetic or semi-synthetic.

6. The composition according to claim 1, further comprising at least one additional active ingredient.

7. The composition of claim 6, wherein the at least one additional active ingredient is selected from the group consisting of: Chondroitin, MSM, Boswellia serrata extract (Aflapin), and combinations thereof.

8.-11. (canceled)

12. A composition according to claim 1, for use in the treatment of at least one disease, condition, symptom or disorder associated with bone and joint diseases.

13. (canceled)

14. A method of treating a disease, condition, symptom or disorder associated with bone and joint diseases in a subject in need thereof; said method comprising administration of a therapeutically effective amount of a composition comprising Cannabidiol (CBD) and Glucosamine and/or a pharmaceutically acceptable glucosamine salt, in a pharmaceutically acceptable dosage form.

15. The method of claim 14, wherein the disease is osteoarthritis.

16. The method of claim 14, wherein the pharmaceutically acceptable glucosamine salt is glucosamine sulfate.

17-18. (canceled)

19. The method according to claim 14, wherein the composition further comprises at least one additional active ingredient.

20. The method of claim 19, wherein the at least one additional active ingredient is selected from the group consisting of Chondroitin, MSM, Boswellia serrata extract (Aflapin), and combinations thereof.

21. The method according to claim 14, wherein the CBD is administered simultaneously as the glucosamine.

22. The method according to claim 14, wherein the CBD is administered separately from the glucosamine.

23. A method of increasing bioavailability of glucosamine in a therapeutic formulation containing an effective amount of glucosamine for treatment of at least one disease, condition, symptom or disorder associated with bone and joint diseases, the method comprising administering Cannabidiol (CBD) in the formulation in a predetermine ratio with the glucosamine.

24. The method of claim 23, wherein the CBD is administered simultaneously as the glucosamine.

25. The method of claim 23, wherein the CBD is administered separately from the glucosamine.

26. The method of claim 23, wherein the at least one disease is osteoarthritis.

27. The method of claim 23, wherein the glucosamine is in the form of a pharmaceutically acceptable salt selected from the group consisting of glucosamine sulfate, glucosamine hydrochloride and N-acetyl-glucosamine.

28-30. (canceled)
